(19) United States
(12) Patent Application Publication
(10) Pub. No.: US 2021/0292819 A1
(43) Pub. Date: Sep. 23, 2021

(54) MULTIPLEX NUCLEIC ACID DETECTION

(71) Applicant: Hewlett-Packard Development Company, L.P., Spring, TX (US)

(72) Inventors: Ryan Murphy, Corvallis, OR (US); Alexander Govyadinov, Corvallis, OR (US)

(73) Assignee: Hewlett-Packard Development Company, L.P., Spring, TX (US)

(21) Appl. No.: 17/059,773

(22) PCT Filed: Dec. 13, 2018
(86) PCT No.: PCT/US2018/065473

§ 371 Date: Nov. 30, 2020

(51) Int. Cl. C12Q 1/6825; C12Q 1/6837; G01N 27/327

(52) U.S. Cl. CPC C12Q 1/6825; G01N 27/3277; C12Q 1/6837

(57) ABSTRACT

A multiplex nucleic acid detection system includes an electrochemical cell and a nucleic acid amplifying fluid. The electrochemical cell includes a reference electrode, a first surface-modified redox electrode including a first working redox electrode with a first surface-attached nucleic acid oligomer attached to the first working electrode, a second surface-modified redox electrode including a second working electrode with a second surface-attached nucleic acid oligomer attached to the second working electrode that is different than the first surface-attached nucleic acid oligomer, and a counter-electrode. The nucleic acid amplifying fluid in this example includes a first dispersed nucleic acid oligomer having a complimentary reverse orientation relative to the first surface-attached nucleic acid oligomer, a second dispersed nucleic acid oligomer having a complimentary reverse orientation relative to the second surface-attached nucleic acid oligomer, and a redox-active compound having affinity to the nucleic acid.

### BACKGROUND

Nucleic acid amplification and detection is a technique utilized in research, medical diagnostics, and forensic testing. The ability to amplify a small quantity of a sample of a nucleic acid to generate copies of the nucleic acid in the sample can permit research, medical diagnostic, and forensic tests that would not otherwise be permissible from the small quantity of the sample, for example.

### BRIEF DESCRIPTION OF THE DRAWINGS

FIG. 1 graphically illustrates an example multiplex nucleic acid detection system in accordance with the present disclosure;

FIG. 2A graphically illustrates an interdigitated example multiplex nucleic acid detection system in accordance with the present disclosure;

FIG. 2B graphically illustrates an interdigitated example multiplex nucleic acid detection system with six surface-modified redox electrodes and four counter-electrodes in accordance with the present disclosure;

FIG. 3A graphically illustrates an interdigitated example multiplex nucleic acid detection system with three surface-modified redox electrodes and two counter-electrodes in accordance with the present disclosure;

FIG. 3B graphically illustrates an interdigitated example multiplex nucleic acid detection system with three surface-modified redox electrodes and four counter-electrodes in accordance with the present disclosure;

FIG. 4 is a flow diagram illustrating an example method of nucleic acid amplification detection in accordance with the present disclosure; and

FIG. 5. graphically illustrates voltammetry scans of an electrical signal generated from a nucleic acid detection system in accordance with an example of the present disclosure.

### DETAILED DESCRIPTION

Nucleic acid amplification can include denaturing, annealing, and extending nucleic acid chains. During denaturing an increased temperature can cause hydrogen bonds between bases in a double-stranded nucleic acid sample to break apart resulting in two single strands realized from a formerly double-stranded nucleic acid. During annealing, the heated sample can then be cooled, enabling single stranded nucleic acid oligomers, such as primers, to attach to the complimentary nitrogen bases on the single strands of the nucleic acid. During extending of the nucleic acid chain the temperature may be increased, for example, to enable a polymerase enzyme to extend the nucleic acid strand by adding nucleic acid bases. In real-time nucleic acid amplification, the amplification of a nucleic acid sample is monitored during the amplification. This can permit the quantification of nucleic acid concentration, gene expression, and sequencing; however, simultaneous amplification and quantification of multiple nucleic acids can be costly, time intensive, or in some instances, unachievable.

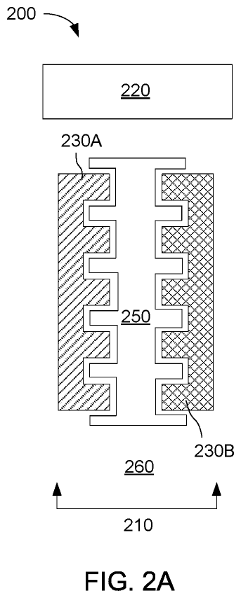

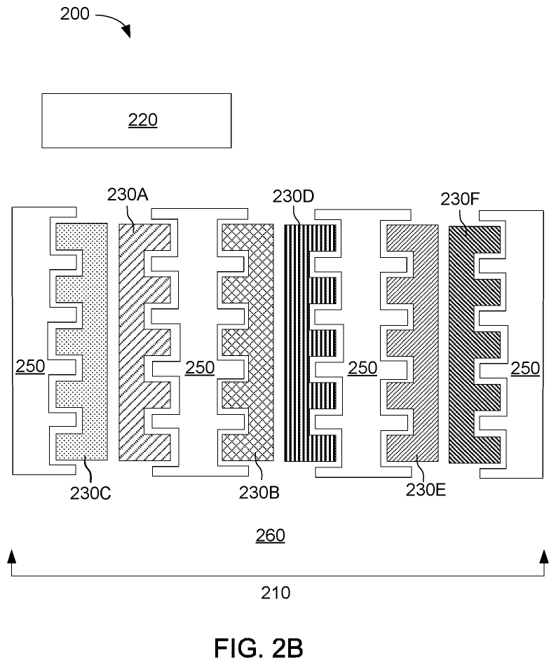

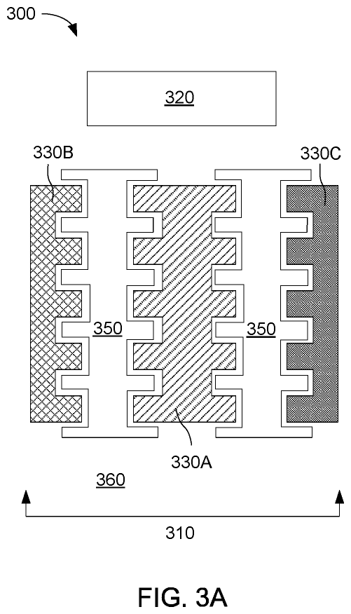

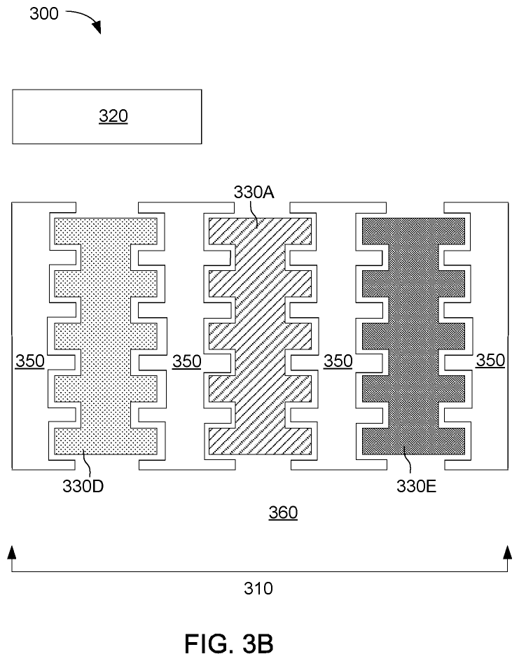

In accordance with examples of the present disclosure, a multiplex nucleic acid detection system includes an electrochemical cell and a nucleic acid amplifying fluid. The electrochemical cell includes a reference electrode, a first surface-modified redox electrode including a first working redox electrode with a first surface-attached nucleic acid oligomer attached to the first working electrode, a second surface-modified redox electrode including a second working electrode with a second surface-attached nucleic acid oligomer attached to the second working electrode and that is different than the first surface-attached nucleic acid oligomer, and a common counter-electrode relative to the first surface-modified redox electrode, the second surface-modified redox electrode, or to both the first and the second surface-modified redox electrode. The nucleic acid amplifying fluid in this example includes a first dispersed nucleic acid oligomer having a complimentary reverse orientation relative to the first surface-attached nucleic acid oligomer, a second dispersed nucleic acid oligomer having a complimentary reverse orientation relative to the second surface-attached nucleic acid oligomer, and a redox-active compound having affinity to the nucleic acid. In one example, the redox-active compound can include hexaamineruthenium (III) chloride, 2-pyridin-2-ylpyridine; tetraoxoosmium, methylene blue, osmium bipyridyl, or a combination thereof. In another example, the counter-electrode is a common counter-electrode for both the first surface-modified redox electrode and the second surface-modified redox electrode. The first and second surface-modified redox electrodes can be interdigitated with respect to the common counter-electrode, for example. By “interdigitated,” this can include any structure pair of multiple electrodes where the electrodes include protrusions that extend into spaces of one another leaving space therebetween, whether that straight finger-like interdigitated electrodes (such as that shown in FIGS. 2 and 3, curved finger-like interdigitated electrodes, spiral interdigitated electrodes, etc. In another example, the nucleic acid amplifying fluid can further include ribose or deoxyribose nucleoside triphosphates and a polymerase enzyme. The reference electrode, the counter-electrode, and the surface-modified redox electrode can independently include material selected from silver, gold, carbon, diamond, diamond-like carbon, platinum, fluorine-doped tin oxide, indium tin oxide, bismuth-doped tin oxide, zinc tin oxide, tantalum tin oxide, strontium calcium copper oxide, bismuth strontium calcium copper oxide, yttrium barium copper oxide, or a combination thereof. Furthermore, the first surface-attached nucleic acid oligomer can be adhered to the first working electrode by an amine functionalized aminopropyltriethoysilane, an amine functionalized glutaraldehyde, or a combination thereof, and the second surface-attached nucleic acid oligomer can be adhered to the second working electrode by an amine functionalized aminopropyltriethoysilane, an amine functionalized glutaraldehyde, or a combination thereof. The electrochemical cell can be integrated on a microfluidic chip as part of a lab-on-a-chip device, for example. The detection system can also further include a third surface-modified redox electrode including a third working electrode with a third surface-attached nucleic acid oligomer that is different than one or both of the first surface-attached nucleic acid oligomer or the second surface-attached nucleic acid oligomer, and in some examples, a second counter-electrode relative to the second surface-modified redox electrode, the third surface-modified redox electrode, or to both the second and the third surface-modified redox electrode.

A multiplex nucleic acid detection system in another example includes an electrochemical cell and a nucleic acid amplifying fluid. The electrochemical cell includes a reference electrode, an array of surface-modified redox electrodes, wherein individual surface-modified redox electrodes include a working redox electrode with a surface-attached nucleic acid oligomer attached its respective working redox electrode, and a common counter-electrode relative to multiple surface-modified redox electrodes of the array. The nucleic acid amplifying fluid includes a dispersed nucleic acid oligomer having a complimentary reverse orientation relative to one or more of the surface-attached nucleic acid oligomers, and a redox-active compound having affinity to the nucleic acid. The array of surface-modified redox electrodes can include from 2 to n surface-modified redox electrodes, and the common counter-electrode can be included in the electrochemical cell array at from 1 to n+1 counter-electrodes relative to the surface-modified redox electrodes. “n” can be, for example, an integer such as 6, 10, 15, 20, 50, 100, etc. As a point of clarification, there can be many surface-modified redox electrodes and many counter-electrodes, but that does not mean that the various redox electrodes include a different nucleic acid oligomer bound to a surface of the various working redox electrodes. They can be unique, as shown in FIG. 2B (with 6 unique surface-modified redox electrodes), or there may be from 2 to 20, 2 to 12, 2 to 6, etc., different types of nucleic acid oligomers, even if there are more working redox electrodes in the system.

In another example, a method of multiplex nucleic acid detection includes loading a nucleic acid amplifying fluid into an electrochemical cell, and amplifying a nucleic acid from within the electrochemical cell using the nucleic acid amplifying fluid. The electrochemical cell includes a reference electrode, an array of surface-modified redox electrodes that individually include a working redox electrode with a surface-attached nucleic acid oligomer attached to its respective working redox electrode, and a counter-electrode relative to multiple surface-modified redox electrodes of the array. The nucleic acid amplifying fluid includes dispersed nucleic acid oligomer having a complementary reverse orientation with respect to one or more of the surface-attached nucleic acid oligomers, and a redox-active compound having affinity to double stranded nucleic acids. The method also includes detecting the nucleic acid bonded to the nucleic acid oligomer on the surface-modified redox electrode based on an electrical signal from the redox-active compound bound associated with the nucleic acid having an affinity for the surface-modified redox electrode. In one example, the method further can include measuring a real time strength of an electrochemical response generated at the surface-modified redox electrode during the amplifying of the nucleic acid. The method can also further include thermal cycling the nucleic acid amplifying fluid within the electrochemical cell for in situ nucleic acid amplification.

When discussing the multiplex nucleic acid detection systems and the method of nucleic acid amplification detection herein, these discussions can be considered applicable to one another whether or not they are explicitly discussed in the context of that example. Thus, for example, when discussing a reference electrode with respect to a multiplex nucleic acid detection system, such disclosure is also relevant to and directly supported in the context of the single and double-stranded multiplex nucleic acid detection systems, alternative multiplexing detection systems, and the method of multiplex nucleic acid detection, etc. Terms used herein will take on their ordinary meaning in the relevant technical field unless specified otherwise. In some instances, there are terms defined more specifically throughout the specification or included at the end of the present specification, and thus, these terms can have a meaning as described herein.

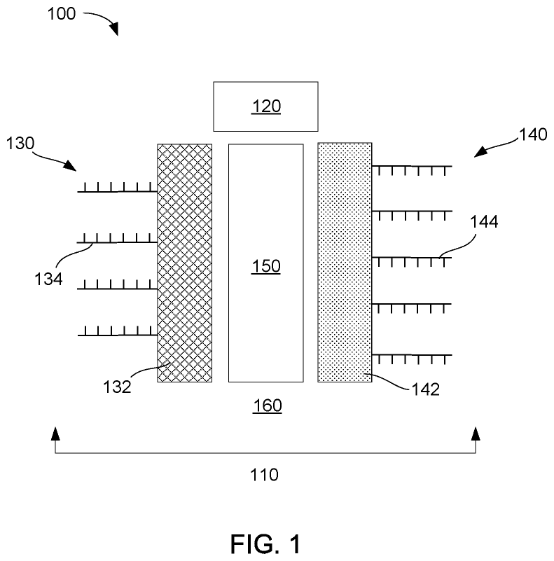

In accordance with examples of the present disclosure, a multiplex nucleic acid detection system 100 is shown in FIG. 1. In one example, the system can include an electrochemical cell 110 and nucleic acid fluid 160, such as an amplifying fluid or portion thereof, master mix fluid of reagent(s) and carrier or portion thereof, etc., which may be packaged in the electrochemical cell or can be co-packaged with the electrochemical cell in a separate container to be added to the system at the time of use. The term “fluid” herein refers to liquids, including liquids with solvated compounds and/or dispersed compounds carried by the liquid as a dispersion. The electrochemical cell can include a reference electrode 120, a first surface-modified redox electrode 130, a second surface-modified redox electrode 140, and a counter-electrode 150 relative to the first surface-modified redox electrode, the second surface-modified redox electrode, or to both the first surface-modified redox electrode and the second surface-modified redox electrode. The first surface-modified redox electrode can include a working redox electrode 132 that can have a first surface-attached nucleic acid oligomer 134. The second surface-modified redox electrode can include a working redox electrode 142 that can have a second surface-attached nucleic acid oligomer 144. The nucleic acid amplifying fluid can include a first dispersed nucleic acid oligomer reverse to the first surface-attached nucleic acid oligomer, a second dispersed nucleic acid oligomer reverse to the second surface-attached nucleic acid oligomer, and a redox-active compound having an affinity to the nucleic acid, e.g., double-stranded nucleic acid. The redox-active compound can have an affinity to nucleic acids that may be formed on the surface-modified redox electrodes. Other compounds that may be in the nucleic acid amplifying fluid include deoxyribose nucleoside triphosphates, polymerase enzymes, magnesium salt, buffer, glycerol, BSA, polysaccharides, other than polymerase enzymes, etc.

In other examples, multiplex nucleic acid detection systems 200 are shown in FIGS. 2A and 2B, and can include an electrochemical cell array 210 and nucleic acid amplifying fluid 260, which may be packaged in the electrochemical cell or can be co-packaged with the electrochemical cell in a separate container to be added to the system at the time of use. The electrochemical cell array can include a reference electrode 220, counter-electrodes 250 that may be common to multiple surface-modified redox electrodes 230A-B (FIG. 2A) or 20A-F (FIG. 2B) of an array of surface-modified redox electrodes. Note that if FIG. 2B, the reference electrode 220, the six electrically separate surface-modified redox electrodes 230, and the four counter-electrodes 250 include the sub-assembly or sub-electrochemical cell array that includes the reference electrode, one of the counter-electrodes, and surface-modified redox electrodes 230A and 230B from FIG. 2A. The individual surface-modified redox electrodes can include a surface-attached nucleic acid oligomer that can be different from other surface-attached nucleic acid oligomers on other surface-modified redox electrodes in the array (as indicated in the figure by different cross-hatching). The surface-modified redox electrodes can be interdigitated, as shown with finger-like interdigitated, but could have other interdigitated configurations, e.g., curved fingers, spiral, etc. The nucleic acid amplifying fluid 360 can include a first dispersed nucleic acid oligomer reverse to the first surface-attached nucleic acid oligomer, a second dispersed nucleic acid oligomer reverse to the second surface-attached nucleic acid oligomer, and a redox-active compound having an affinity to the nucleic acid, e.g., double-stranded DNA. The redox-active compound can have an affinity to nucleic acids that may be formed on the surface-modified redox electrodes. Other compounds that may be in the nucleic acid amplifying fluid include deoxyribose nucleoside triphosphates, polymerase enzymes, magnesium salt, buffer, glycerol, BSA, polysaccharides, other than polymerase enzymes etc.

In yet another example, as shown in FIGS. 3A and 3B, two example electrochemical cell arrays 310 of the multiplex nucleic acid detection systems 300 are shown, that can include the electrochemical cell array and nucleic acid amplifying fluid 360, which may be packaged in the electrochemical cell or can be co-packaged with the electrochemical cell in a separate container to be added to the system at the time of use. Both examples include a reference electrode 320, multiple counter-electrodes 350 relative to multiple surface-modified redox electrodes 330A, 330B, 330C (FIG. 3A) or 330A, 330D, 330E (FIG. 3B). Note that if FIG. 3B, the reference electrode, the multiple surface-modified redox electrodes, and the four counter-electrodes 350 include the presence of a sub-assembly or sub-electrochemical cell array shown in FIG. 3A, which namely includes the reference electrode, two (of the four) counter-electrodes, and surface-modified redox electrode 330A in either electrochemical cell array assembly. In both of these examples, a bidirectional interdigitated redox electrode is included (one in FIG. 3A at 330A and three is FIG. 3B at 330A, 330D, and 330D). With respect to FIG. 3B compared to the array shown in FIG. 2B, as there is no electrical isolation separating the bidirectionally oriented interdigitated fingers, as is the case shown in FIG. 2B (see channel between adjacent redox electrodes), there are only three surface-modified redox electrode in this example compared to six redox electrodes in FIG. 2B. The various surface-modified redox electrodes 330A-E can include a working redox electrode that has a surface-attached nucleic acid oligomer (See FIG. 1 illustrating the working electrode and the surfaced-attached nucleic acid oligomer, for example). The individual surface-modified redox electrodes can have a different surface-attached nucleic acid oligomer than the other surface-modified redox electrodes in the array (as indicated in the figure by different hatching), or there can be fewer different oligomers with some working electrodes having the same oligomer as other electrodes. In this arrangement (FIG. 2B), there are counter-electrodes on either end of the electrochemical cell array, unlike FIG. 2A where the electrodes at either end are surface-modified redox electrodes. Again, the surface-modified redox electrodes can be interdigitated, as shown with finger-like interdigitated, but could have other interdigitated configurations, e.g., curved fingers, spiral, etc. The nucleic acid amplifying fluid 360 can include a first dispersed nucleic acid oligomer reverse to the first surface-attached nucleic acid oligomer, a second dispersed nucleic acid oligomer reverse to the second surface-attached nucleic acid oligomer, and a redox-active compound having an affinity to the nucleic acid, e.g., double-stranded DNA. The redox-active compound can have an affinity to nucleic acids that may be formed on the surface-modified redox electrodes. Other compounds that may be in the nucleic acid amplifying fluid include deoxyribose nucleoside triphosphates, polymerase enzymes, magnesium salt, buffer, glycerol, BSA, polysaccharides, other than polymerase enzymes etc.

Regardless of the configuration, in some examples, an electrochemical cell or electrochemical cell array can be integrated on a microfluidic chip, such as a lab-on-a-chip device. In one example, the microfluidic chip can be an integrated point of care diagnostic device, such as an in vitro diagnostic point of care device. In another example, the microfluidic chip can include a microfluidic channel, microfluidic chamber, electrical chemical cell, electrochemical sensor, heating element, temperature controller, detector, or a combination thereof. If included, microfluidic channels and/or chambers can be arranged in parallel, in series, or a combination thereof.

With respect to the electrodes, the reference electrode can allow for the measurement of an electrochemical response generated at a surface-modified redox electrode. In further detail, an electrochemical cell or electrochemical cell array with three types of electrodes, including the reference electrode, can allow for the determination of an electrochemical signature of a material using a working electrode (or array of working electrodes) and a counter-electrode (or multiple counter-electrodes, rather than mere current flow values between the two electrode types. The reference electrode can be constructed of to have a stable and known electrode potential, and can be used in a half-cell, or in some other cell configuration, to provide a reference for evaluating a material based on its electrochemical signature.

In one example, the reference electrode can have stable electrical properties. The range of the electrical potential for the reference cell can be from −500 mV to 500 mV compared to a standard hydrogen electrode, which is defined as having 0 V. In one example, the reference electrode can include a material that can be independently selected from silver, gold, carbon, diamond, diamond-like carbon, platinum, silver chloride, fluorine-doped tin oxide, indium tin oxide, bismuth-doped tin oxide, zinc tin oxide, tantalum tin oxide, an alloy thereof, or a combination thereof. In another example, the reference electrode can include a material independently selected from silver, gold, carbon, diamond, diamond-like carbon, platinum, an alloy, or a combination thereof. In yet another example, the reference electrode can include a material independently selected from fluorine-doped tin oxide, indium tin oxide, bismuth-doped tin oxide, zinc tin oxide, tantalum tin oxide, silver chloride, an alloy thereof, or a combination thereof. In a further example, the reference electrode can include a ceramic material that can be independently selected from strontium calcium copper oxide (SrₓCa_{y}Cu₂O where x+y is about 1, bismuth strontium calcium copper oxide (Bi₂Sr₂Ca₂Cu₃O₁₀), yttrium barium copper oxide (YBaCuO), or a combination thereof.

The Ag or Ag/AgCl reference electrode can have a thickness that can range from 100 Å to 100 μm. In one example, the reference electrode can have a surface area that can range from 25 μm² to 1,000 μm². In another example, the reference electrode can be square or rectangular in shape and can be from 1 μm to 20,000 μm in length in one direction and from 1 μm to 1,000 μm in width in a perpendicular direction to the length. In other examples, the reference electrode can of other non-rectangular shapes.

The surface-modified redox electrode as described herein can also be referred to as a first surface-modified redox electrode, a second surface-modified redox electrode, and so forth, or as part of an array of surface-modified redox electrodes, for example. In one example, an individual surface-modified redox electrode can differ from another surface-modified redox electrode in an electrochemical cell or an electrochemical cell array based on the surface-attached nucleic acid oligomer, for example. In another example, the structure of an individual surface-modified redox electrode can differ from a structure of another surface-modified redox electrode in an electrochemical cell or an electrochemical cell array based on the dimensional structure or material structure of the redox electrode. Both structure and surface-attached nucleic acid oligomer can likewise be different from one surface-modified redox electrode to another, for example.

The surface-modified redox electrode include a working redox electrode (as the substrate) with a surface-attached nucleic acid oligomer (as the attached oligomer, e.g., a primer. The surface-modified redox electrode can be structurally configured to be capable of developing an electrical signal in response to a redox-active compound, e.g., intercalating with a double-stranded nucleic acid or by some other chemically attractive or attaching mechanism. In one example, a surface-modified redox electrode can be operated at electrical potentials ranging from −700 mV to 1100 mV vs. a silver/silver chloride reference electrode.

A surface-modified redox electrode can include a material that can be independently selected from gold, carbon, diamond, diamond-like carbon, platinum, fluorine-doped tin oxide, indium tin oxide, bismuth-doped tin oxide, zinc tin oxide, tantalum tin oxide, an alloy thereof, or a combination thereof. In another example, a surface-modified redox electrode can include a material independently selected from gold, carbon, diamond, diamond-like carbon, platinum, an alloy, or a combination thereof. In yet another example, a surface-modified redox electrode can include a material independently selected from fluorine-doped tin oxide, indium tin oxide, bismuth-doped tin oxide, zinc tin oxide, tantalum tin oxide, an alloy thereof, or a combination thereof. In a further example, a surface-modified redox electrode can include a ceramic material that can be independently selected from strontium calcium copper oxide, bismuth strontium calcium copper oxide (Bi₂Sr₂Ca₂Cu₃O₁₀), yttrium barium copper oxide (YBaCuO), or a combination thereof.

A surface-attached nucleic acid oligomer can be attached to the working redox electrode by an oligomer amine functionalized aminopropyltriethoysilane, an oligomer amine functionalized glutaraldehyde, or a combination thereof. In one example, the first surface-attached nucleic acid oligomer can be independently adhered to the first working redox electrode and to the second working redox electrode by an oligomer amine functionalized aminopropyltriethoysilane, an oligomer amine functionalized glutaraldehyde, or a combination thereof.

An oligomer that can be attached to a working redox electrode can be selected to correspond to a nucleic acid to be amplified and detected in a sample. In one example, the oligomer can be a nucleic acid primer. In another example, the oligomer can include a primer for mRNA, RNA, DNA, or a combination thereof.

The structure of a surface-modified redox electrode can be regular or irregular in shape. In one example, a surface-modified redox electrode can be square, rectangular, or some other shape, including any of a number of interdigitated shapes or configurations relative to an adjacently positioned counter-electrode, for example. In one example, a surface-modified redox electrode can be rectangular as shown in FIG. 1. In yet another example, a surface-modified redox electrode can be interdigitated as shown in FIGS. 2 and 3, with finger-like interdigitated protrusions from multiple opposing electrodes (e.g., working and counter-electrodes) nested within recesses of the opposing electrode (without touching). In another example, the interdigitation can be some other shapes, such as having curved protrusions and nested in inversely shaped recesses (not touching), or having an interdigitated spiral configuration, for example. In one example, surface-modified redox electrodes can be interdigitated in any manner where protrusions from the working electrode can be interlockingly arranged with respect to its counter-electrode, without physical contact therebetween.

The electrochemical cell or electrochemical cell array can also include a counter-electrode or multiple counter-electrodes. A counter-electrode can close an electric circuit and balance a reaction occurring at the working redox electrode. In one example, a counter-electrode can act electrically in concert with (with opposing charges) with respect to the surface-modified redox electrode to thereby provide a circuit over which a current can be measured and evaluated for an electrical signature with respect to the reference electrode. In some examples, the counter-electrode can be a common counter-electrode (a shared counter electrode) for multiple redox working electrodes, e.g., the first redox working electrode and the second working redox electrode.

A counter-electrode can include a material that can be independently selected from gold, carbon, diamond, diamond-like carbon, platinum, fluorine-doped tin oxide, indium tin oxide, bismuth-doped tin oxide, zinc tin oxide, tantalum tin oxide, an alloy thereof, or a combination thereof. In another example, a counter-electrode can include a material independently selected from gold, carbon, diamond, diamond-like carbon, platinum, an alloy, or a combination thereof. In yet another example, a counter-electrode can include a material independently selected from fluorine-doped tin oxide, indium tin oxide, bismuth-doped tin oxide, zinc tin oxide, tantalum tin oxide, an alloy thereof, or a combination thereof. In a further example, a counter-electrode can include a ceramic material that can be independently selected from strontium calcium copper oxide, bismuth strontium calcium copper oxide (Bi₂Sr₂Ca₂Cu₃O₁₀), yttrium barium copper oxide (YBaCuO), or a combination thereof.

In examples herein, a redox-active compound can have an affinity for the nucleic acid, which can be concentrated at or near a working redox electrode during operation of the electrochemical cell or cell array. The redox-active compound can be, for example, an intercalating chemical, which can insert itself in spaces between the hydrogen bonds of a double-stranded nucleic acid. The energy for intercalation can be furnished by a redox chemical reaction. Thus, the intercalation can increase the concentration of the redox-active compound, thus increasing the electrical signal. In still further detail, energy value to add or remove an electron (chemically change) the redox-active compound can be measured.

In one example, the redox-active compound can include hexaamineruthenium (III) chloride, 2-pyridin-2-ylpyridine; tetraoxoosmium, methylene blue, or combinations thereof. In one example, the redox-active compound can include hexaamineruthenium (III) chloride. In another example, the redox-active compound can include methylene blue.

The redox-active compound can be disposed in a nucleic acid amplifying fluid, such as a master mix, or in one example, a polymerase chain reaction (PCR) master mix. The nucleic acid amplifying fluid can further include a first dispersed nucleic acid oligomer reverse to the first surface-attached nucleic acid oligomer, and a second dispersed nucleic acid oligomer reverse to the second surface-attached nucleic acid oligomer. Other compounds that may be present can include deoxyribose nucleoside triphosphates, polymerase enzyme(s), magnesium halide (chloride), solvent such as DNAse free water, dimethyl sulfoxide (DMSO), glycerol, and/or bovine serum albumin (BSA), for example.

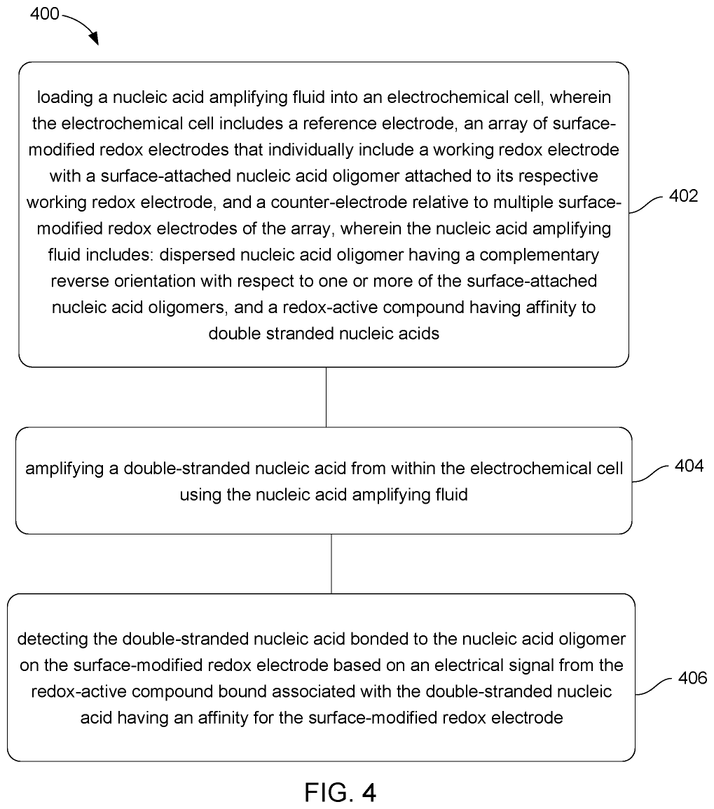

A flow diagram of an example method of nucleic acid amplification detection 400 is shown in FIG. 4. The method can include loading 402 a nucleic acid amplifying fluid into an electrochemical cell, and amplifying 404 a nucleic acid from within the electrochemical cell using the nucleic acid amplifying fluid. The electrochemical cell includes a reference electrode, an array of surface-modified redox electrodes that individually include a working redox electrode with a surface-attached nucleic acid oligomer attached to its respective working redox electrode, and a counter-electrode relative to multiple surface-modified redox electrodes of the array. The nucleic acid amplifying fluid includes dispersed nucleic acid oligomer having a complementary reverse orientation with respect to one or more of the surface-attached nucleic acid oligomers, and a redox-active compound having affinity to nucleic acids. The method also includes detecting 406 the nucleic acid bonded to the nucleic acid oligomer on the surface-modified redox electrode based on an electrical signal from the redox-active compound bound associated with the nucleic acid having an affinity for the surface-modified redox electrode. In one example, the method further can include measuring a real time strength of an electrochemical response generated at the surface-modified redox electrode during the amplifying of the nucleic acid. The method can also further include thermal cycling the nucleic acid amplifying fluid within the electrochemical cell for in situ nucleic acid amplification.

In further detail, loading the nucleic acid amplifying solution can include injecting from 10 pL to 10 μL of a nucleic acid amplifying solution into the electrochemical cell or cell array. In some examples, injecting can include pipetting, fluid ejecting, or the like. Amplifying a nucleic acid within the electrochemical cell can include polymerase chain reaction (PCR), strand displacement assay, transcription mediated assay, isothermal amplification, loop mediated isothermal amplification, reverse-transcription loop mediated isothermal amplification, nucleic acid sequence based amplification, recombinase polymerase amplification, or multiple displacement amplification. In one example, amplification can include polymerase chain reaction, such as reverse transcription polymerase chain reaction.

During amplification, hydrogen bonds on a double-stranded nucleic acid can be denatured and two single strands of nucleic acid can be produced. The single strands of nucleic acid can bind to a corresponding oligomer that can be attached to a working redox electrode. Deoxyribose nucleoside triphosphates can bind to nucleic acid bases on the single strand of nucleic acid attached to the corresponding oligomer on the working redox electrode. A redox-active compound having affinity to double-stranded nucleic acids can intercalate in spaces of the double-stranded nucleic acid. The intercalation can result from a redox reaction, which can thereby generate an electrical signal.

Detection of the nucleic acid bonded to the nucleic acid oligomer on the surface-modified redox electrode can include measuring an electrical signal generated by the redox-active compound. In one example, the signal can be measured by a potentiostat. In some examples, the method can further include analyzing the electrical signal detected by comparing it to a reference signal measured at the reference electrode.

In some examples, the method can further include measuring a real time strength of an electrochemical response generated at the surface-modified redox electrode during the amplifying of the double-stranded nucleic acid. The measuring can include detecting an electrical signal continuously, or detecting an electrical signal at set intervals. For example, the measuring can include detecting an electrical signal at intervals such as a time within the range of every 30 seconds to every fifteen minutes, every 1 minute to every 10 minutes, every two minutes to every five minutes, or some other suitable time frame based on the measurements to be taken.

In yet another example, the method can further include thermal cycling the nucleic acid amplifying fluid within the electrochemical cell for in situ nucleic acid amplification. Thermal cycling can include denaturing, annealing, and extending nucleic acid chains based on temperature changes. A “thermal cycle” can be defined by the temperatures used for denaturing, annealing, and extending phases of the amplification. An increased temperature can cause hydrogen bonds between bases in a double-stranded nucleic acid sample to break apart resulting in two single strands or nucleic acid realized from a formerly double stranded nucleic acid. During annealing, the heated sample can then be cooled, enabling single stranded nucleic acid oligomers, such as primers, on a surface-modified redox electrode to attach to the complimentary nitrogen bases on the single strands of the nucleic acid. During extending of the nucleic acid chain the temperature may be increased, for example, to enable a polymerase enzyme to extend the nucleic acid strand by adding nucleic acid bases. Regardless of the sequence or heating and cooling and the temperatures that are reached during the heating and cooling phases, thermal cycling can be repeated until a desired number of nucleic acid copies, e.g., DNA, are formed, which can for example take from about 10 to about 70 thermal cycles, or 20 to 50 thermal cycles, or 10 to 30 thermal cycles, in many instances.

In some examples, temperature changes can be controlled by an internal heating element of a microfluidic chip. In other examples, temperature changes can be controlled by an external heating element. In yet other examples, the method can include cooling utilizing an internal cooling element or an external cooling device.

As used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though the individual member of the list is identified as a separate and unique member. Thus, no individual member of such list should be construed as a de facto equivalent of any other member of the same list based on their presentation in a common group without indications to the contrary.

Concentrations, dimensions, amounts, and other numerical data may be presented herein in a range format. A range format is used merely for convenience and brevity and should be interpreted flexibly to include the numerical values explicitly recited as the limits of the range, as well as to include all the individual numerical values or sub-ranges encompassed within that range as the individual numerical value and/or sub-range is explicitly recited. For example, a weight ratio range of about 1 wt % to about 20 wt % should be interpreted to include the explicitly recited limits of 1 wt % and 20 wt % and to include individual weights such as about 2 wt %, about 11 wt %, about 14 wt %, and sub-ranges such as about 10 wt % to about 20 wt %, about 5 wt % to about 15 wt %, etc.

### EXAMPLES

The following illustrates examples of the present disclosure. However, it is to be understood that the following are illustrative of the application of the principles of the present disclosure. Numerous modifications and alternative compositions, methods, and systems may be devised without departing from the present disclosure.

### Example 1—Electrochemical Cell Analysis

An electrochemical cell including a silver/silver chloride pseudo-reference electrode, indium tin oxide counter-electrode, and an indium tin oxide redox working electrode functionalized with an oligomer having a known single strand DNA nucleic acid sequence on a surface thereof were arranged on a silicon chip. A square wave voltammetry scan was ran at a 150 mV/s scan rate a 100 mV amplitude using an Anapot Potentiostat, available from Zimmer and Peacock. A nucleic acid amplifying fluid including hybridized oligomer chains with nitrogenous base sequences suitable for base pairing (A with T and C with G) with the oligomer chains attached to the working electrode to form double-stranded DNA was loaded onto a silicon chip. The nucleic acid amplifying fluid also included 25 μM hexaamineruthenium (III) chloride (a redox-active compound having an affinity to double-stranded nucleic acid). The square wave voltammeter was used to scan an electrical signal generated at the redox working electrode.

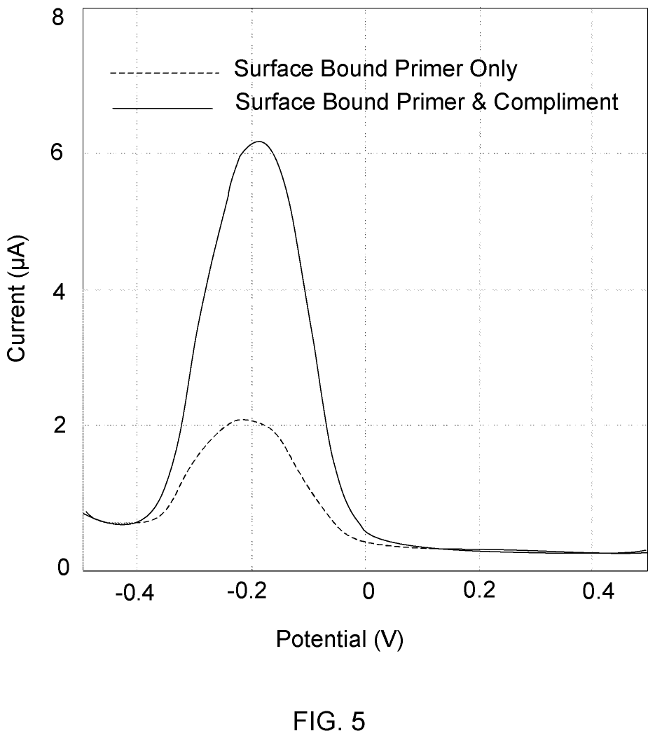

The results of the voltammetry scans are graphically presented in FIG. 5. The surface-modified redox working electrode generated a lower redox response before the oligomer on the surface-modified redox working electrode was hybridized with the reverse nucleic acid sequence. This example demonstrated that a redox-active compound can intercalate with a nucleic acid sequence bound to a working redox electrode, thereby generating an electrical signal at the surface of the electrode. By knowing a sequence of the oligomer attached to the working redox electrode, the binding rate of a particular sequence can be monitored to identify specific nucleic acid sequences in a sample during amplification.

### Example 2—Electrochemical Cell Array Analysis

An electrochemical cell array including an silver/silver chloride pseudo-reference electrode, two indium tin oxide common counter-electrodes, and four indium tin oxide redox working electrodes functionalized with an oligomer are arranged on a silicon chip. The redox working electrodes and counter-electrodes can have an interdigitated structures relative to one another, and the counter-electrodes can be arranged as common counter-electrodes for multiple redox working electrodes. The four redox working electrodes can have different oligomers attached thereto. A square wave voltammetry scan can be ran at a 150 mV/s scan rate and a 100 mV amplitude using a voltammeter.

A nucleic acid amplifying fluid that can include a nucleic acid amplifying fluid containing hybridized oligomer chains with nitrogenous base sequences suitable for base pairing (A with T and C with G) with the various oligomer chains attached to the various working electrodes to form double-stranded DNA can be loaded into this electrochemical cell array. A second square wave voltammetry scan can be conducted to measure an electrical signal generated at the redox working electrode. The electrochemical cell array can allow for parallel data acquisition of multiple nucleic acid strands during the amplification process.

While the present technology has been described with reference to certain examples, various modifications, changes, omissions, and substitutions can be made without departing from the disclosure. It is intended, therefore, that the disclosure be limited by the scope of the following claims.

What is claimed is:

1. A multiplex nucleic acid detection system, comprising:
an electrochemical cell including:
a reference electrode,
a first surface-modified redox electrode including a first working redox electrode with a first surface-attached nucleic acid oligomer attached to the first working electrode,
a second surface-modified redox electrode including a second working electrode with a second surface-attached nucleic acid oligomer attached to the second working electrode and that is different than the first surface-attached nucleic acid oligomer, and
a counter-electrode relative to the first surface-modified redox electrode, the second surface-modified redox electrode, or to both the first and the second surface-modified redox electrode; and
a nucleic acid amplifying fluid including a first dispersed nucleic acid oligomer having a complimentary reverse orientation relative to the first surface-attached nucleic acid oligomer, a second dispersed nucleic acid oligomer having a complimentary reverse orientation relative to the second surface-attached nucleic acid oligomer, and a redox-active compound having affinity to the nucleic acid.

2. The multiplex nucleic acid detection system of claim 1, wherein the redox-active compound includes hexaamineruthenium (III) chloride, 2-pyridin-2-ylpyridinet; tetraoxoosmium, methylene blue, or a combination thereof.

3. The multiplex nucleic acid detection system of claim 1, wherein the counter-electrode is a common counter-electrode for both the first surface-modified redox electrode and the second surface-modified redox electrode.

4. The multiplex nucleic acid detection system of claim 3, wherein the first and second surface-modified redox electrodes are interdigitated with respect to the common counter-electrode.

5. The nucleic acid amplification detection system of claim 1, wherein the nucleic acid amplifying fluid further comprises ribose or deoxyribose nucleoside triphosphates, and a polymerase enzyme.

6. The multiplex nucleic acid detection system of claim 1, wherein the reference electrode, the counter-electrode, and the surface-modified redox electrode include material independently selected from gold, carbon, diamond, diamond-like carbon, platinum, fluorine-doped tin oxide, indium tin oxide, bismuth-doped tin oxide, zinc tin oxide, tantalum tin oxide, strontium calcium copper oxide, bismuth strontium calcium copper oxide, yttrium barium copper oxide, or a combination thereof.

7. The multiplex nucleic acid detection system of claim 1, wherein the first surface-attached nucleic acid oligomer is adhered to the first working electrode by an amine functionalized aminopropyltriethoysilane, an amine functionalized glutaraldehyde, or a combination thereof, and wherein the second surface-attached nucleic acid oligomer is adhered to the second working electrode by an amine functionalized aminopropyltriethoysilane, an amine functionalized glutaraldehyde, or a combination thereof.

8. The multiplex nucleic acid detection system of claim 1, wherein the electrochemical cell is integrated on a microfluidic chip as part of a lab-on-a-chip device.

9. The multiplex nucleic acid detection system of claim 1, further comprising a third surface-modified redox electrode including a third working electrode with a third surface-attached nucleic acid oligomer that is different than one or both of the first surface-attached nucleic acid oligomer or the second surface-attached nucleic acid oligomer.

10. The multiplex nucleic acid detection system of claim 9, further comprising a second counter-electrode relative to the second surface-modified redox electrode, the third surface-modified redox electrode, or to both the second and the third surface-modified redox electrode.

11. A multiplex nucleic acid detection system, comprising:
an electrochemical cell including:
a reference electrode,
an array of surface-modified redox electrodes, wherein individual surface-modified redox electrodes include a working redox electrode with a surface-attached nucleic acid oligomer attached its respective working redox electrode, and
a common counter-electrode relative to multiple surface-modified redox electrodes of the array, wherein the multiple surface-modified redox electrodes are interdigitated with respect to the common counter-electrode; and
a nucleic acid amplifying fluid including a dispersed nucleic acid oligomer having a complimentary reverse orientation relative to one or more of the surface-attached nucleic acid oligomers, and a redox-active compound having affinity to the nucleic acid.

12. The multiplex nucleic acid detection system of claim 11, wherein the array of surface-modified redox electrodes include from 2 to n surface-modified redox electrodes, wherein n is 100, and wherein the counter-electrodes are included in an array at from 1 to n+1.

13. A method of multiplex nucleic acid detection, comprising:
loading a nucleic acid amplifying fluid into an electrochemical cell, wherein the electrochemical cell includes a reference electrode, an array of surface-modified redox electrodes that individually include a working redox electrode with a surface-attached nucleic acid oligomer attached to its respective working redox electrode, and a counter-electrode relative to multiple surface-modified redox electrodes of the array, wherein the nucleic acid amplifying fluid includes: dispersed nucleic acid oligomer having a complementary reverse orientation with respect to one or more of the surface-attached nucleic acid oligomers, and a redox-active compound having affinity to double stranded nucleic acids;
amplifying a nucleic acid from within the electrochemical cell using the nucleic acid amplifying fluid; and
detecting the nucleic acid bonded to the nucleic acid oligomer on the surface-modified redox electrode based on an electrical signal from the redox-active compound bound associated with the nucleic acid having an affinity for the surface-modified redox electrode.

14. The method of rapid thermal cycling of claim 13, further comprising measuring a real time strength of an electrochemical response generated at the surface-modified redox electrode during the amplifying of the nucleic acid.

15. The method of rapid thermal cycling of claim 14, further comprising thermal cycling the nucleic acid amplifying fluid within the electrochemical cell for in situ nucleic acid amplification.
